(19) United States
(12) Patent Application Publication
(10) Pub. No.: US 2003/0220530 A1
(43) Pub. Date: Nov. 27, 2003

(54) Process and apparatus for preparing olefins

(75) Inventors: Heinz Boelt, Wolfratshausen (DE); Helmut Fritz, Muenchen (DE); Stephan Glanz, Neubiberg (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: LINDE AKTIENGESELLSCHAFT, Wiesbaden (DE)

(21) Appl. No.: 10/409,072

(22) Filed: Apr. 9, 2003

(30) Foreign Application Priority Data
Apr. 22, 2002 (DE) 102 17 863.1

(51) Int. Cl. C07C 4/02; B01J 8/02

(52) U.S. Cl. 585/648; 585/651; 422/190; 422/198

(57) ABSTRACT

A hydrocarbon-containing feed, e.g. naphtha, is converted in a treatment plant (1), in particular a refinery or olefin plant, into desired olefins, e.g. ethylene and propylene. At least part of the fractions comprising relatively long-chain olefins, in particular olefins having at least four carbon atoms, obtained from the treatment plant (1) is passed to an olefin conversion stage (12). In the olefin conversion stage (12), for example, a catalytic reactor, in particular a fixed-bed reactor, at least part of the relatively long-chain olefins is converted into relatively shorter-chain olefins. To increase the yield of desired olefins, the olefin conversion stage (12) is preceded by a paraffin/olefin separation stage (23) wherein the feed stream is separated into a paraffin stream, e.g., a stream comprising butane and pentane, and an olefin stream, e.g,. a stream comprising butene and pentene, e.g., by extractive distillation. The paraffins obtained are recirculated to the treatment plant (1), e.g., to the cracking furnace (3) of the olefin plant, and the olefins are fed to the olefin conversion stage (12).

The invention relates to a process for preparing olefins from a hydrocarbon-containing feed, where the feed is fed to a treatment plant, in particular a refinery or olefin plant, in which various hydrocarbon-containing fractions are produced, at least part of the fractions comprising relatively long-chain olefins, in particular olefins having at least four carbon atoms, is fed to an olefin conversion stage in which at least part of the relatively long-chain olefins is converted into shorter-chain olefins that are at least partly recirculated to the treatment plant, and also to an apparatus for carrying out the process.

The market for chemical raw materials has shown an increasing demand for propylene in recent years. In the past, propylene has been produced mainly as by-product in olefin plants, in particular ethylene plants. Since the future demand for propylene will no longer be able to be covered by this route alone, processes for increasing the propylene yield in olefin plants have already been developed. These processes are based essentially on the principle of converting relatively long-chain olefins, e.g. those having from four to six carbon atoms, obtained in olefin plants into shorter-chain olefins having, for example, two and three carbon atoms, in particular into propylene (three carbon atoms). Here, use is usually made of a catalyst which effects the appropriate conversion of the olefins. Such processes are known under the names “Propylur process” (cf. Hydrocarbon Engineering, May 1999, pages 66 to 68) and “Meta-4 process” (cf. Petrochemicals PTQ Spring 1997, pages 109 to 115) and can be summarized under the collective term olefin conversion processes. The economics of such processes depend greatly on the achieved yield of the desired olefins.

It is an object of the present invention to configure a process of the type mentioned at the outset and an apparatus for carrying out the process in such a way that an increase in the yield of the desired olefins having up to three carbon atoms is made possible.

Upon further study of the specification and appended claims, further objects and advantages of this invention will become apparent to those skilled in the art.

According to the invention, a process aspect is achieved by the olefin conversion stage being preceded by a paraffin/olefin separation stage in which olefins and paraffins are separated from one another, with at least part of the paraffins being fed to the treatment plant or taken off and passed to another use and at least part of the olefins being fed to the olefin conversion stage.

The treatment plant for the preparation of olefins is usually an olefin plant comprising a cracking furnace for cracking the hydrocarbons and a downstream fractionation stage for separating the cracking products into individual fractions. However, it is also possible to utilize particular product streams from a refinery for the preparation of olefins, for example so as to provide additional utilization opportunities for the refinery.

The idea on which the invention is based is to separate the hydrocarbon mixture which is to be recirculated to the olefin conversion stage into paraffins and olefins beforehand and to pass only the olefins to the olefin conversion stage. In this way, the yield of desired olefins can be increased. The paraffins which have been separated off form a suitable feed for the cracking furnace of the olefin plant, which is why they are preferably fed to the cracking furnace. Alternatively or in addition, the paraffins can also be taken off and passed to another use. Overall, this improves both the economics of the total olefin plant or refinery and the yield of desired olefins.

In a particularly preferred embodiment of the invention, the olefin conversion stage is followed by a conversion product fractionation stage in which at least one fraction which comprises hydrocarbons having four and/or five carbon atoms is produced and is recirculated to the olefin conversion stage. In addition, at least one second fraction comprising hydrocarbons having at least five carbon atoms is produced and is taken off. The conversion product fractionation stage thus produces a total of three fractions, namely a fraction which comprises hydrocarbons having not more than three carbon atoms and is recirculated to the treatment plant, e.g. to the fractionation stage of the olefin plant, a fraction comprising hydrocarbons having four and/or five carbon atoms and a fraction comprising hydrocarbons having at least five carbon atoms. The olefins obtained in the paraffin/olefin separation stage may either be fed directly to the olefin conversion stage or passed firstly to the conversion product fractionation stage, bypassing the olefin conversion stage, so that they subsequently enter the olefin conversion stage via olefin recirculation.

A further development of the concept of the invention provides for separation into at least three fractions taking place in the paraffin/olefin separation stage, with a first fraction comprising paraffins having four and/or five carbon atoms being fed to the treatment plant, e.g the cracking furnace of the olefin plant, a second fraction comprising olefins having four and/or five carbon atoms being fed to the olefin conversion stage, and a third fraction comprising hydrocarbons having at least five carbon atoms being taken off. In this variant of the invention, the conversion product fractionation stage can be simpler, since only one fraction comprising hydrocarbons having not more than three carbon atoms has to be separated off. In this case, the paraffin/olefin separation stage is designed so that separation into the three product streams mentioned is made possible.

The paraffin/olefin separation stage advantageously comprises an extractive distillation. This extractive distillation is preferably configured so that the three product streams mentioned can be produced.

The olefin conversion stage preferably comprises a catalytic reaction, e.g. catalytic cracking, of the relatively long-chain hydrocarbons to form shorter-chain hydrocarbons. Such catalytic conversion processes are known per se from the prior art. The invention further provides an apparatus for preparing olefins from a hydrocarbon-containing feed, which comprises a treatment plant, in particular a refinery or olefin plant, which has a fractionation apparatus for producing various hydrocarbon-containing fractions, with the fractionation apparatus being connected to an olefin conversion apparatus for converting relatively long-chain olefins into shorter-chain olefins.

An apparatus aspect of the invention is achieved by the olefin conversion apparatus being preceded by a paraffin/olefin separation apparatus for separating olefins and paraffins which is connected via a paraffin line to the treatment plant and via an olefin line to the olefin conversion apparatus.

The treatment plant for preparing the olefins can be a refinery or olefin plant. In the case of an olefin plant, it is provided with a cracking furnace for cracking the hydrocarbons which is followed by a fractionation apparatus for separating the cracking products into fractions. The paraffin/olefin separation apparatus is in this case connected via the paraffin line to the cracking furnace of the olefin plant.

The paraffin/olefin separation apparatus is advantageously equipped with distillation columns designed for extractive distillation. At least two distillation columns are provided, with one serving to separate paraffins and olefins and the second distillation column being configured as a regeneration column for recovering the extractant.

According to a further development of the concept of the invention, at least three distillation columns are provided, as a result of which it is additionally possible to separate off hydrocarbons having more than five carbon atoms. In this case, the conversion product fractionation apparatus can be simpler, since only the short-chain hydrocarbons having fewer than three carbon atoms have to be separated off.

The olefin conversion apparatus preferably has a catalytic reactor, in particular a fixed-bed reactor. As catalysts, it is possible to use the types of catalyst known from the prior art, in particular a zeolite catalyst.

The invention makes it possible, in particular, to achieve a significant increase in the propylene yield in olefin plants or refineries for a justifiable engineering outlay. The additional process step of paraffin/olefin separation substantially increases the yield of target products in olefin plants. At the same time, circulated streams are minimized so that the specific capital costs for propylene production are reduced. This compensates for the additional capital investment for the additional process step of paraffin/olefin separation. Overall, the economics of the olefin plant or the refinery are improved.

The entire disclosure of all applications, patents and publications, cited above and below, and of corresponding German Application No. 102 17 863.1, filed Apr. 22, 2002, is hereby incorporated by reference.

The invention is illustrated below with the aid of a comparison of the prior art with the illustrative examples of the invention shown schematically in the figures.

### BRIEF DESCRIPTION OF THE DRAWINGS

Various other features and attendant advantages of the present invention will be more fully appreciated as the same becomes better understood when considered in conjunction with the accompanying drawings, in which like reference characters designate the same or similar parts throughout the several views, and wherein:

FIG. 1 shows a flow diagram of an olefin plant having an integrated olefin conversion stage according to the prior art;

FIG. 2 shows an olefin plant having an integrated olefin conversion stage and an upstream paraffin/olefin separation stage;

FIG. 3 shows an olefin plant having an integrated olefin conversion stage with conversion product fractionation stage and an upstream paraffin/olefin separation stage, with direct olefin feed to the olefin conversion stage;

FIG. 4 as FIG. 3, but with indirect olefin feed to the olefin conversion stage;

FIG. 5 shows a flow diagram of an olefin plant having an integrated olefin conversion stage with conversion product fractionation apparatus and an upstream paraffin/olefin separation stage with integrated fractionation; and

FIGS. 6 and 7 show various embodiments of the paraffin/olefin separation stage.

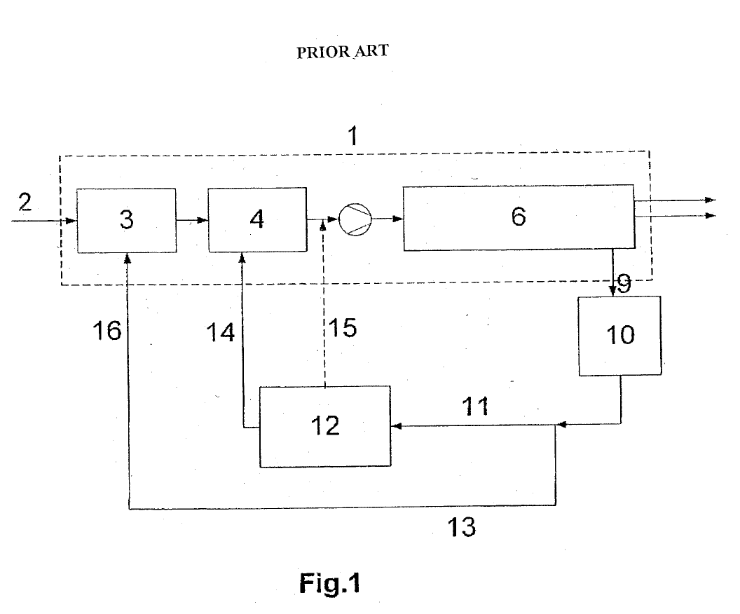

The olefin plant 1 according to the prior art shown in FIG. 1 is operated using naphtha which is fed via line 2 to a cracking furnace 3. It is also possible for the plant to be provided with a plurality of cracking furnaces which convert the naphtha into a cracking gas which is passed to a water scrub 4 and, by means of a compressor 5, to a fractionation stage 6. In the fractionation stage 6, olefins, pyrolysis gasoline, pyrolysis oil and light gases are taken off via lines 7 and 8. Hydrocarbons having four or five carbon atoms obtained in the fractionation stage 6 are fed via line 9 to a diene/acetylene removal unit 10. The remaining paraffins and olefins having four and/or five carbon atoms are passed via line 11 to an olefin conversion stage 12. The effulent streams in lines 11 and 13 both contain paraffins and olefins. The olefin conversion stage 12 is usually configured as a catalytic fixed-bed reactor and converts the relatively long-chain olefins (C₄/C₅-olefins) into shorter-chain olefins (C₂-C₄-olefins. Butanes discharged via line 13 are supplied directly as feed to the cracking furnace 3 where they are cracked to form olefins and by-products. The olefins having from two to four carbon atoms produced in the olefin conversion stage 12 and gasoline and light gases obtained are conveyed via line 14 to the water scrub 4 of the olefin plant 1. A substream can also be conveyed directly via line 15 to upstream of the pump 5. This arrangement represents the simplest variant disclosed in the prior art for increasing the propylene yield in olefin plants.

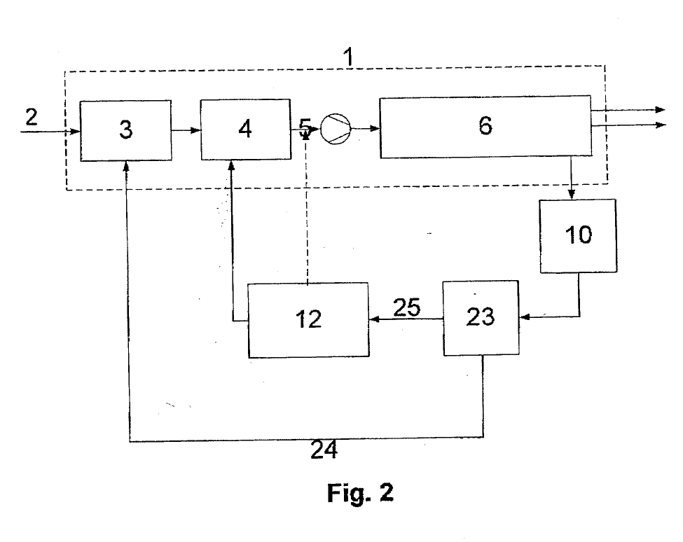

FIG. 2 shows an embodiment of the invention which is based on the plant according to the prior art shown in FIG. 1. The same plant components are denoted by the same reference numerals. This variant of the invention differs from the prior art in that a paraffin/olefin separation stage 23 is provided upstream of the olefin conversion stage 12. This additional process step serves to separate the paraffins from the olefins. While the paraffins (butane, pentane) are conveyed as feed to the cracking furnace 3 of the olefin plant 1 via line 24, the olefins (butene, pentene) are fed via an olefin line 25 to the olefin conversion stage 12. This enables a significant increase in the propylene yield of the olefin plant to be achieved.

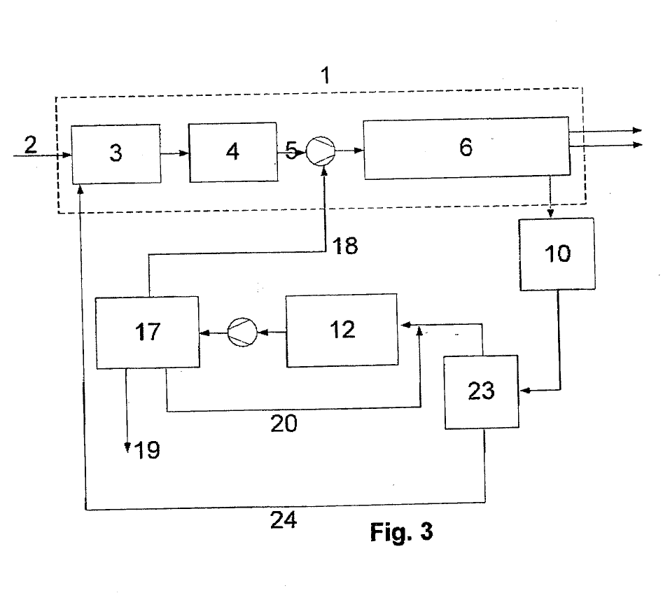

The embodiment shown in FIG. 3 differs from that shown in FIG. 2 in that the olefin conversion stage 12 is followed by a conversion product fractionation stage 17 in which the hydrocarbons produced in the olefin conversion stage 12 are separated into various fractions. The hydrocarbons having not more than three carbon atoms are recirculated via line 18 directly to the fractionation stage 6 of the olefin plant 1, while the relatively long-chain hydrocarbons having at least five carbon atoms and gasoline product are discharged via line 19, and the hydrocarbons having four and/or five carbon atoms are taken off from the conversion product fractionation stage 17 via line 20 and recirculated to the olefin conversion stage 12.

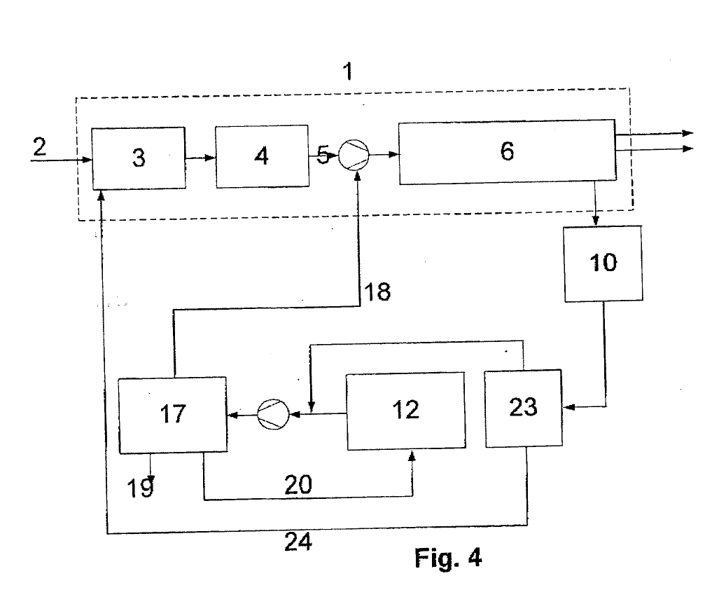

FIG. 4 shows an alternative to the embodiment shown in FIG. 3, in which the olefins obtained in the paraffin/olefin separation stage 23 are not fed directly to the olefin conversion stage 12 but instead indirectly via the conversion product fractionation apparatus and the olefin recirculation line 20.

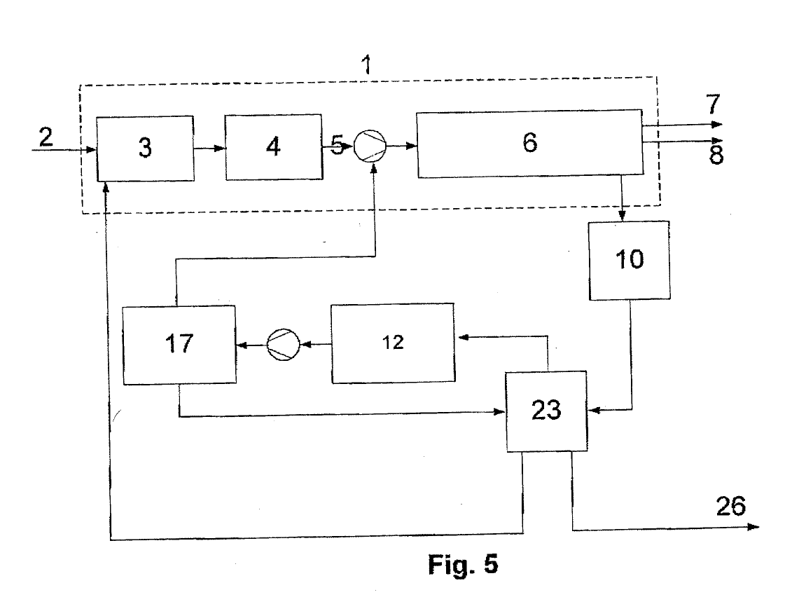

FIG. 5 shows another variant of the invention, in which the paraffin/olefin separation stage 23 is designed so as to serve simultaneously as fractionation stage. This makes it possible to omit the removal of hydrocarbons having more than five carbon atoms in the conversion product fractionation stage 17 as shown in FIG. 3. These relatively long-chain hydrocarbons are thus not discharged via line 19 as shown in FIG. 3 but instead from the propylene/olefin separation stage 23 via line 26. The other process steps correspond to the variant shown in FIG. 3.

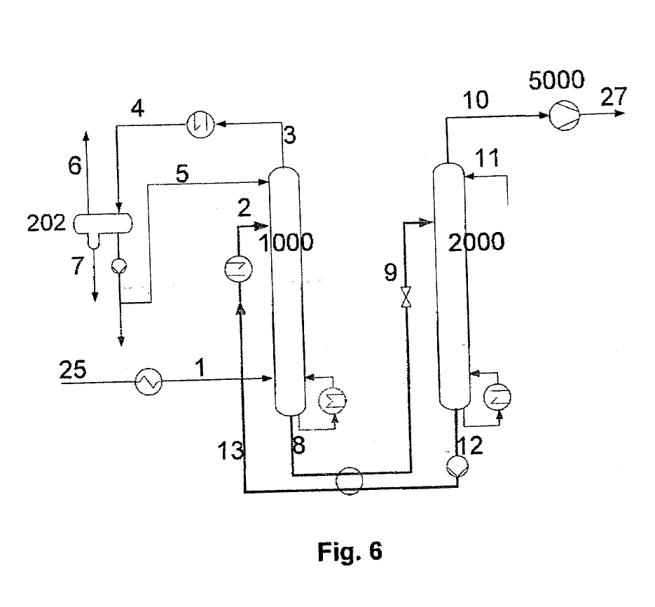

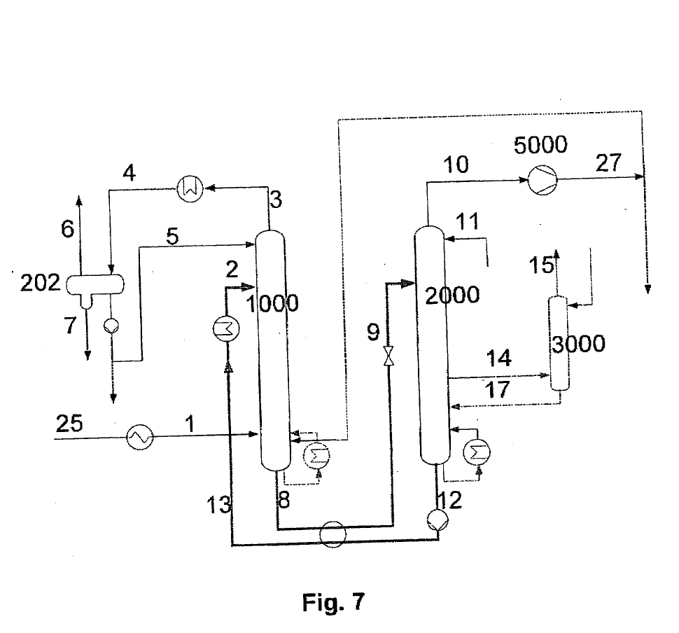

FIGS. 6 and 7 show variants of the paraffin/olefin separation stage in detail. The paraffin/olefin separation stage is in each case configured as an extractive distillation.

The paraffin/olefin separation stage shown in FIG. 6 is supplied via lines 25 and 1 with a mixture of butane, pentane, butene and pentene which is taken off from the conversion product fractionation apparatus. This mixture is introduced into a first distillation column 1000. The distillation column 1000 is operated using an extractant which is introduced via lines 12, 13 and 2 into the upper part of the distillation column 1000. The extractant can comprise the following substances: NMP (N-methylpyrrolidone), DMF (dimethylformamide), NFM (N-formylmorpholine), acetone, acetonitrile, furfural, DMAC (dimethylacetamide), sulpholane, diethylene glycol (glycol mixtures) or dimethyl sulphoxide. Solvent mixtures can also be used, and water or methanol can be employed as additives. The paraffins butane and/or pentane are taken off from the top of the distillation column 1000 via line 3 and are finally recirculated via line 6 to the cracking furnace of the olefin plant. Part of these paraffins is recirculated via line 5 to the distillation column 1000 for backscrubbing. Water can be separated off via line 7. The olefins butene and pentene obtained at the bottom of the distillation column 1000 are conveyed via line 8 and a heat exchanger 104 and line 9 to a second distillation column 2000 which serves as regeneration column. For backscrubbing, water or an external runback stream is fed via line 11 to the top of the distillation column 2000. The olefins butene and pentene are finally taken off from the top of the distillation column 2000 via line 10 and recirculated via a compressor 5000 and line 27 to the olefin conversion stage. The distillation column 1000 is, in the present example, operated at a pressure of 9 bar which is sufficient for further processing of the paraffins in the cracking furnace. If the paraffins are intended only for liquefaction or for introduction into a fuel gas supply, a pressure in the distillation column 1000 of from 4 to 6 bar is sufficient. The pressure at which the distillation column 2000 is operated is as low as possible in order to avoid decomposition of the extractant. For this reason, the pressure in the present example is from 1 to 4 bar, in particular less than 2 bar. If an auxiliary (e.g. higher hydrocarbons) is introduced, the pressure should be high enough for condensation to be achieved or for the top product to be able to be taken off in gaseous form. In this case, the pressure is from 2 to 6 bar (condensation: 4-5 bar, gaseous top product: >3 bar). The temperatures in the distillation columns 1000 and 2000 are matched to the respective pressures and the separation task to be achieved. In the regeneration column 2000, a boiling temperature which is sufficiently below the decomposition temperatures of the solvents is set at the bottom.

In FIG. 7, the same plant components as in FIG. 6 are denoted by the same reference numerals. The variant depicted in FIG. 7 differs from that shown in FIG. 6 by an additional measure for removing acetylene which comprises a third distillation column 3000 which is connected via lines 14 and 17 to the distillation column 2000. Acetylene is taken off from the top of the distillation column 2000 via line 15. Backscrubbing with water or an external runback stream takes place via line 16.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A process for preparing olefins from a hydrocarbon-containing feed, comprising:
introducing hydrocarbon-containing feed to a treatment plant (1) wherein at least one hydrocarbon-containing fraction at least part comprising relatively long-chain olefins is produced;
feeding same hydrocarbon-containing fraction to an olefin conversion stage (12) in which at least part of the relatively long-chain olefins is converted into relatively shorter-chain olefins; and
recirculating at least part of said relatively shorter-chain olefins to the treatment plant (1);
wherein said olefin conversion stage (12) is preceded by a paraffin/olefin separation stage (23), in which olefins and paraffins are separated from one another, at least part of the separated paraffins are fed to the treatment plant (1) or taken off and passed to another use, and at least part of the separated olefins are fed to the olefin conversion stage (12).

2. A process according to claim 1, wherein treatment plant (1) is an olefin plant comprising a cracking furnace (3) for cracking the hydrocarbons and a fractionation stage (6) for separating the cracking products into fractions, and at least part of the paraffins separated off from the olefins in the olefin separation stage (23) is fed to the olefin plant.

3. A process according to claim 1, wherein the olefin conversion stage (12) is followed by a conversion product fractionation stage (17) from which are removed at least one first fraction, comprising hydrocarbons having four and/or five carbon atoms which is recirculated to the olefin conversion stage (12), and at least one second fraction which comprises hydrocarbons having at least five carbon atoms.

4. A process according to claim 2, wherein the olefin conversion stage (12) is followed by a conversion product fractionation stage (17) from which are removed at least one first fraction, comprising hydrocarbons having four and/or five carbon atoms which is recirculated to the olefin conversion stage (12), and at least one second fraction which comprises hydrocarbons having at least five carbon atoms.

5. A process according to claim 3, wherein the olefins obtained from the paraffin/olefin separation stage (23) are fed directly to the conversion product fractionation stage (17), bypassing the olefin conversion stage (12).

6. A process according to claim 4, wherein the olefins obtained from the paraffin/olefin separation stage (23) are fed directly to the conversion product fractionation stage (17), bypassing the olefin conversion stage (12).

7. A process according to claim 1, wherein the stream fed to the paraffin/olefin separation stage (23) is separated into at least three fractions: a first fraction comprising paraffins having four and/or five carbon atoms being fed to the treatment plant (1); a second fraction comprising olefins having four and/or five carbon atoms being fed to the olefin conversion stage (12); and a third fraction comprising hydrocarbons having at least five carbon atoms.

8. A process according to claim 2, wherein the stream fed to the paraffin/olefin separation stage (23) is separated into at least three fractions: a first fraction comprising paraffins having four and/or five carbon atoms being fed to the treatment plant (1); a second fraction comprising olefins having four and/or five carbon atoms being fed to the olefin conversion stage (12); and a third fraction comprising hydrocarbons having at least five carbon atoms.

9. A process according to any of claims 1 to 8, wherein the paraffin/olefin separation stage (23) comprises an extractive distillation.

10. A process according to any of claims 1 to 9, wherein the olefin conversion stage (12) comprises a catalytic conversion of relatively long-chain hydrocarbons into relatively shorter-chain hydrocarbons.

11. An apparatus for preparing olefins from a hydrocarbon-containing feed, comprising:
a treatment plant (1) comprising a fractionator (6) for producing various hydrocarbon-containing fractions, said fractionator (6) being in fluid communication with an olefin conversion apparatus (12) for converting relatively long-chain olefins into shorter-chain olefins, wherein said olefin conversion apparatus (12) is preceded by a paraffin/olefin separation apparatus (23) for separating olefins and paraffins;
a paraffin discharge line in fluid communication with said paraffin/olefin separation apparatus (23) and the treatment plant (1); and an olefin line in fluid communication with said paraffin/olefin separation apparatus (23) and the olefin conversion apparatus (12).

12. An apparatus according to claim 11, wherein the treatment plant (1) is an olefin plant comprising a cracking furnace (3) for cracking the hydrocarbons and said fractionator apparatus (6) for separating the cracking products into fractions, and the paraffin/olefin separation apparatus (23) is in fluid communication with said cracking furnace (3) via said paraffin line.

13. An apparatus according to claim 11, wherein said paraffin/olefin separation apparatus (23) comprises at least one extractive distillation column.

14. An apparatus according to claim 12, wherein said paraffin/olefin separation apparatus (23) comprises at least one extractive distillation column.

15. An apparatus according to claim 11, wherein olefin conversion apparatus (12) comprises a catalytic reactor.

16. An apparatus according to claim 12, wherein olefin conversion apparatus (12) comprises a catalytic reactor.

17. An apparatus according to claim 13, wherein olefin conversion apparatus (12) comprises a catalytic reactor.

18. An apparatus according to claim 14, wherein olefin conversion apparatus (12) comprises a catalytic reactor.

19. An apparatus according to claim 15, wherein said catalytic reactor is a fixed-bed reactor.

20. An apparatus according to claim 16, wherein said catalytic reactor is a fixed-bed reactor.

21. An apparatus according to claim 17, wherein said catalytic reactor is a fixed-bed reactor.

22. An apparatus according to claim 18, wherein said catalytic reactor is a fixed-bed reactor.
